(19) United States
(12) Patent Application Publication
(10) Pub. No.: US 2022/0000666 A1
(43) Pub. Date: Jan. 6, 2022

(54) USE OF A TREATMENT DEVICE WITH A LASER FOR CORRECTING AN EYE TISSUE, AND A METHOD FOR PROVIDING CONTROL DATA FOR A LASER FOR CORRECTING AN EYE TISSUE

(71) Applicant: SCHWIND eye-tech-solutions GmbH, Kleinostheim (DE)

(72) Inventor: Pascal NAUBEREIT, Aschaffenburg (DE)

(21) Appl. No.: 17/363,412

(22) Filed: Jun. 30, 2021

(30) Foreign Application Priority Data
Jul. 1, 2020 (DE) 10 2020 117 393.8

(51) Int. Cl. A61F 9/008

(52) U.S. Cl. CPC A61F 9/00827; A61F 2009/00872

(57) ABSTRACT

Use of a treatment apparatus is disclosed for cut-free transfer of a tissue of a correction area of a human or animal eye from a determined actual state into an ascertained desired state. The treatment apparatus includes a fiber laser device, which includes a fiber oscillator and/or a fiber amplifier. In addition, a method is disclosed for providing control data of a fiber laser device for a correction of the eye tissue as well as to the corresponding apparatuses.

The invention relates to the use of a treatment apparatus with at least one laser for cut-free or incision-free transfer of a tissue of a correction area of a human or animal eye from a determined actual state into an ascertained desired state. In this context, a treatment apparatus is understood to be a device or a group of devices comprising a laser, for example a laser system.

Non-surgical ophthalmological methods such as for example URIC (“laser-induced refractive index change”, laser-induced refractive change of the refractive index) are used to change a refractive index of a human or animal eye without a lenticule having to be cut from the cornea.

Such methods can also be referred to as “cut-free”, since the eye tissue is not cut for example by a laser. Thus, “cut-free” methods do not include opening tissue and changing the shape of the tissue. Heretofore, a solid-state laser is for example used for LIRIC. Other non-surgical or cut-free methods are cross-linking applications, by which not only a visual disorder can be treated, but by which diverse other syndromes can be treated, for example a keratoconus. Many systems for such non-surgical methods have to be water-cooled due to great waste heat. They are very high-maintenance or required parameters are not achieved. Other laser types have different advantages, but also disadvantages. One disadvantage is often the lack of flexibility with respect to the parameter space, a required stability of the parameters or a necessary freedom from maintenance.

An object underlying the invention is increasing a flexibility of parameter space and parameter stability and freedom from maintenance for cut-free ophthalmological methods, thus for non-surgical methods.

The set object is solved by the use according to the invention, the method according to the invention and the apparatuses according to the invention according to the coordinate claims. Advantageous further embodiments are given by the dependent claims.

The invention is based on the idea of using a fiber laser device instead of for example a solid-state laser for cut-free ophthalmological methods. An appliance, an appliance group or appliance component is understood by a fiber laser device, which includes a fiber laser, in other words, a fiber oscillator and/or a fiber amplifier. A fiber laser device combines many advantages of the individual laser types without having the corresponding disadvantages, wherefore the use of a fiber laser device for cut-free ophthalmological methods yields considerable advantages. A fiber laser offers the required flexibility with respect to the parameter space (in particular for example variable repetition rate and variable/short pulse duration), the required stability of the parameters (in particular for example pulse energy, pulse duration, repetition rate and pulse shape), and an increased freedom from maintenance (for example air cooling (“air-cooled”) and a long lifetime). The flexibility with respect to the parameter space arises in that many parameters can be easier achieved with a fiber laser. Therein, a fiber oscillator and a fiber amplifier can for example be encompassed by the fiber laser device according to the invention, but also for example a fiber oscillator and a solid-state amplifier. By the employment of the fiber laser device, which is heretofore only used for removing lenticules in the prior art, thus for surgical and thereby invasive methods, a non-surgical or cut-free method is optimized by the invention.

A first aspect relates to the use of a treatment apparatus with at least one laser, thus an ophthalmological laser, in particular a laser, which is heretofore used in the eye surgical method, in a non-surgical method, thus in the use for cut-free transfer of a tissue of a correction area of a human or animal eye from a determined actual state into an ascertained desired state.

For example, the actual state of the eye can be a visual disorder, but also another syndrome, for example a keratoconus. The ascertained desired state can then for example be a calculated correction of the refractive index, thus for example a reduced or eliminated visual disorder, or a cornea with a reduced or removed keratoconus.

The use according to the invention provides that the treatment apparatus includes a fiber laser device, which includes a fiber oscillator and/or a fiber amplifier.

The above mentioned advantages result.

Optionally, the use can provide that the ascertained desired state satisfies a preset visual disorder reduction criterion, which presets that the eye with the tissue of the desired state has a visual disorder reduced compared to the determined actual state of the tissue. Such a use can for example induce a chemical process in the eye tissue, in which water, which is bound in collagen of the eye tissue, is released, and thereby the refractive index of the eye changes. By the above mentioned advantages of the fiber laser device, a visual disorder can be reduced in such a use in non-invasive manner or at least in very minimally invasive manner. By the combination with the advantages of the fiber laser device, a treatment can be particularly precisely effected.

The use according to the invention can preferably be effected in a method for laser-induced change of a refractive index (LIRIC) and/or in a cross-linking method.

In a further advantageous configuration of the use according to the invention, the fiber laser device is configured to emit laser pulses in a wavelength range between 300 nanometers (nm) and 1400 nanometers, preferably between 700 nanometers and 1200 nanometers, at a respective pulse duration between one femtosecond (fs) and one nanosecond (ns), preferably between 10 femtoseconds and 10 picoseconds (ps), and a repetition frequency of greater than 10 kilohertz (kHz), preferably between 100 kilohertz and 100 megahertz (MHz). Such a fiber laser device configured as a femtosecond laser is particularly well suitable for treating a cornea and additionally has the advantage that the irradiation of the cornea does not have to be effected in a wavelength range below 300 nanometers. This range is subsumed by the term “deep ultraviolet” in the laser technology. By this embodiment, it is advantageously avoided that an unintended damage to the cornea is effected by these very short-wavelength and high-energy beams. A power density required for an optical breakthrough can be spatially narrowly limited. In particular, the wavelength range between 700 nanometers and 780 nanometers is advantageous.

The object set above is solved by a method for providing control data of a fiber laser device for a correction of an eye tissue, wherein the advantages already mentioned above are achieved. The fiber laser device includes a fiber oscillator and/or a fiber amplifier. Therein, a control device performs the following method steps. An appliance, an appliance component or an appliance group is understood by a control device, which is designed and configured for receiving and evaluating signals as well as for generating control signals. For example, the control device can be configured as a control unit or control chip or computer program.

The control device determines an actual state of a tissue of a correction area of the eye, for example a visual disorder or a keratoconus, optionally also the shape of the keratoconus. For determining the actual state, the control device can for example evaluate data, which describes the visual disorder or the keratoconus, or for example data of an appliance for measuring the cornea.

Based on the determined actual state of the tissue, the control device ascertains a desired state of the tissue, thus for example a desired corneal shape for eliminating or reducing the visual disorder, or for example an area of the cornea as well as the state thereof for changing the refractive index. Alternatively, the desired state can for example describe the shape of the cornea with a reduced or eliminated or removed keratoconus.

The control device provides control data, which describes an operation of the fiber laser device for cut-free transfer of the tissue of the correction area from the determined actual state into the ascertained desired state.

In an embodiment of the method according to the invention, the control device can determine visual disorder data of the human or animal eye, for example a visual disorder in diopters, wherein the visual disorder data can describe a visual disorder. The ascertained desired state of the tissue can then satisfy a preset visual disorder reduction criterion, which presets that the eye with the tissue in the desired state has a visual disorder reduced compared to the determined actual state of the tissue. The advantages were already addressed above.

According to a further embodiment of the method according to the invention, the provided control data can describe a laser-induced change of a refractive index (LIRIC) and/or a cross-linking method.

Preferably, the control device can be configured to cause the fiber laser device to emit laser pulses in a wavelength range between 300 nanometers and 1400 nanometers, preferably between 700 nanometers and 1200 nanometers, at a respective pulse duration between one femtosecond and one nanosecond, preferably between 10 femtoseconds and 10 picoseconds, and a repetition frequency of greater than 10 kilohertz, preferably between 100 kilohertz and 100 megahertz. The advantages were already discussed above.

According to a further embodiment of the method according to the invention, the control device can transmit the provided control data to the fiber laser device of the treatment apparatus. Hereby, the control of the fiber laser device is initiated.

A third aspect of the present invention relates to a control device, which is configured to perform one of the above described embodiments of the method according to the invention. The above cited advantages arise. The control device can for example be configured as a control chip, control unit or application program (“app”). The control device can preferably comprise a processor device and/or a data storage. An appliance or an appliance component for electronic data processing is understood by a processor device. For example, the processor device can comprise at least one microcontroller and/or at least one microprocessor. Preferably, a program code for performing the method according to the invention can be stored on the optional data storage. The program code can then be configured, upon execution by the processor device, to cause the control device to perform one of the above described embodiments of the method according to the invention.

A fourth aspect of the present invention relates to a treatment apparatus with at least one fiber laser device, wherein the treatment apparatus comprises an embodiment of the control device according to the invention. The above described advantages arise.

In a further advantageous configuration of the treatment apparatus according to the invention, the fiber laser device can be suitable to emit laser pulses in a wavelength range between 300 nm and 1400 nm, preferably between 700 nm 30 and 1200 nm, at a respective pulse duration between 1 fs and 1 ns, preferably between 10 fs and 10 ps, and a repetition frequency of greater than 10 kilohertz (kHz), preferably between 100 kHz and 100 megahertz (MHz). The advantages already mentioned above arise.

In further advantageous configurations of the treatment apparatus according to the invention, the control device can comprise at least one storage device for at least temporary storage of at least one control dataset, wherein the control dataset or datasets include control data for positioning and/or for focusing individual laser pulses in the cornea; and can comprise at least one beam device for beam guidance and/or beam shaping and/or beam deflection and/or beam focusing of a laser beam of the laser. Therein, the mentioned control datasets are usually generated based on a measured topography and/or pachymetry and/or morphology of the cornea to be treated and the type of the visual disorder to be corrected.

Further features and the advantages thereof can be taken from the descriptions of the first inventive aspect, wherein advantageous configurations of each inventive aspect are to be regarded as advantageous configurations of the respectively other inventive aspect.

A fifth aspect of the invention relates to a computer program including instructions, which cause the treatment apparatus according to the fourth inventive aspect to execute the method steps according to the second inventive aspect.

A sixth aspect of the invention relates to a computer-readable medium, on which the computer program according to the fifth inventive aspect is stored. Further features and the advantages thereof can be taken from the descriptions of the first to fourth inventive aspects, wherein advantageous configurations of each inventive aspect are to be regarded as advantageous configurations of the respectively other inventive aspect.

Further features of the invention are apparent from the claims, the figures and the description of figures. The features and feature combinations mentioned above in the description as well as the features and feature combinations mentioned below in the description of figures and/or shown in the figures alone are usable not only in the respectively specified combination, but also in other combinations without departing from the scope of the invention. Thus, implementations are also to be considered as encompassed and disclosed by the invention, which are not explicitly shown in the figures and explained, but arise from and can be generated by separated feature combinations from the explained implementations. Implementations and feature combinations are also to be considered as disclosed, which thus do not comprise all of the features of an originally formulated independent claim. Moreover, implementations and feature combinations are to be considered as disclosed, in particular by the implementations set out above, which extend beyond or deviate from the feature combinations set out in the relations of the claims.

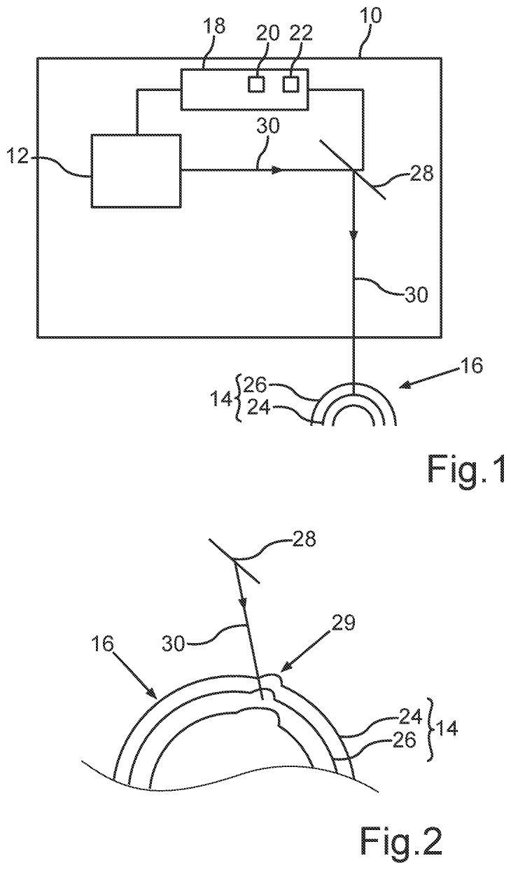

FIG. 1 is a schematic representation of the apparatuses according to the invention, of the use according to the invention and of the method according to the invention.

FIG. 2 is a schematic diagram of the use according to the invention and of the method according to the invention.

In the figures, identical or functionally identical elements are provided with the same reference characters.

FIG. 1 shows a schematic representation of a treatment apparatus 10 with a fiber laser device 12 for example for a cross-linking method and/or a LIRIC. The fiber laser device 12 can for example be employed to initiate a chemical process in a cornea 14 of a human or animal eye 16, in which water is released from collagen of the eye tissue. Preferably, the fiber laser device 12 can comprise a fiber oscillator as well as a solid-state laser amplifier or a fiber laser amplifier.

The illustrated fiber laser device 12 can preferably be a fiber laser, which is formed to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz.

FIG. 1 shows a control device 18 for the fiber laser device 12, which can be formed to control the fiber laser device 12 such that it emits pulsed laser pulses for example in a predefined pattern into the cornea 14. Alternatively, the control device 18 can be a control device 18 external with respect to the treatment apparatus 10. The control device 18 can preferably comprise a storage device 20, for example a hard disk or a memory chip, and/or a processor device 22, which can exemplarily include multiple microprocessors or microcontrollers. The storage device 20 can be for at least temporary storage of at least one control dataset, wherein the control dataset or datasets can include control data for positioning and/or for focusing individual laser pulses in the cornea 14.

The position data and/or focusing data of the individual laser pulses can for example be generated based on a previously measured topography and/or pachymetry and/or the morphology of the cornea 14 and the pathological and/or unnaturally altered correction area for example to be removed or an optical visual disorder correction to be generated exemplarily within a stroma 24 below an epithelium 26 of the eye 16, preferably based on a determined actual geometry of the cornea 14 as the actual state in the correction area and based on an analysis how the eye tissue is to be corrected to for example eliminate or reduce a keratoconus 29 or a visual disorder.

The laser beam 30 generated by the fiber laser device 12 by means of a beam device 28 can be deflected towards a surface of the cornea 14. The beam deflection device is also controlled by the control device 18.

As shown in the example of FIG. 2, the cornea 14 of the eye 16 can for example form a keratoconus 29. Such a keratoconus 29 as the actual state, thus a local steepening of the cornea 14, can form in that the cornea 14 is as thin at this location as the cornea 14 curves outwards by the pressure of the bulk body of the eye 16, thus by the intraocular pressure. A wanted desired state can then be a reduced or even removed or eliminated keratoconus 29. Here, the area, in which the tissue to be corrected is located, is referred to as correction area.

For determining the actual state of the correction area of the eye 16 (method step S1, cf. FIG. 1), the keratoconus 29 can for example be measured, or corresponding data describing the involved tissue layers can be received from the storage device 20 or from a data server (not shown in the figures).

For optionally determining visual disorder data (optional method step S2), which can for example indicate a value in diopters, the control device 18 can for example receive the corresponding data from a data server or the storage device 20, or the data can be determined as a data input.

Optionally, a three-dimensional, preferably digital model of the cornea 14 and/or of the correction area can for example be provided based on the determined actual state. Based on such a digital model, the desired state of the tissue can for example be ascertained (S3).

Based on the determined desired geometry, the control device 18 can now provide, preferably generate, the control data (S4), and transfer it to the fiber laser device 12 (S5).

Overall, the embodiments illustrate how a fiber laser device 12, in particular a fiber laser, can be used for non-surgical applications, thus for cut-free applications.

According to a further embodiment, a fiber laser device 12, in particular a fiber laser, is employed for such cut-free, ophthalmological applications. The fiber laser device 12 combines many advantages of the individual laser types without having the corresponding disadvantages. A fiber laser device 12 is particularly well suitable for cut-free applications.

Accordingly, it will be readily understood by those persons skilled in the art that, in view of the above detailed description of the invention, the present invention is susceptible of broad utility and application. Many adaptations of the present invention other than those herein described, as well as many variations, modifications, and equivalent arrangements will be apparent from or reasonably suggested by the present invention and the above detailed description thereof.

While the present invention is described herein in detail in relation to specific aspects, it is to be understood that this detailed description is only illustrative and exemplary of the present invention and is made merely for purposes of providing a full and enabling disclosure of the present invention and to provide the best mode contemplated by the inventor or inventors of carrying out the invention. The detailed description set forth herein is not intended nor is to be construed to limit the present invention or otherwise to exclude any such other embodiments, adaptations, variations, modifications, and equivalent arrangements of the present invention.

1.-15. (canceled)

16. A treatment apparatus for a correction of an eye tissue comprising:
at least one fiber laser device including a fiber oscillator and/or a fiber amplifier; and
a control device configured to
determine an actual state of a tissue of a correction area of a human or animal eye,
ascertain a desired state of the tissue based on the determined actual state of the tissue, and
provide control data to the at least one fiber laser device that describes an operation of the at least one fiber laser device for cut-free transfer of the tissue of the correction area from the determined actual state into the ascertained desired state.

17. The treatment apparatus according to claim 15, wherein the fiber laser device is suitable to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz.

18. The treatment apparatus according to claim 16, wherein the control device comprises
at least one storage device for at least temporary storage of at least one control dataset, wherein the at least one control dataset includes control data for positioning and/or for focusing individual laser pulses in the cornea, and
at least one beam device for beam guidance and/or beam shaping and/or beam deflection and/or beam focusing of a laser beam of the at least one fiber laser device.

19. The treatment apparatus according to claim 16, wherein the control device is configured to determine visual disorder data of the human or animal eye, which describes a visual disorder, wherein the ascertained desired state of the tissue satisfies a preset visual disorder reduction criterion, which presets that the human or animal eye with the tissue in the desired state has a visual disorder reduced compared to the determined actual state of the tissue.

20. The treatment apparatus according to claim 16, wherein the operation described by the control data utilizes a laser-induced change of a refractive index (LIRIC) and/or a cross-linking method.

21. The treatment apparatus according to claim 16, wherein the control device transmits the control data to the at least one fiber laser device of the treatment apparatus.

22. A method for providing control data to a fiber laser device for a correction of an eye tissue, the fiber laser device having a fiber oscillator and/or a fiber amplifier, and a control device, the control device configured for performing the method by
determining an actual state of a tissue of a correction area of the eye,
ascertaining a desired state of the tissue based on the determined actual state of the tissue, and
providing control data to the fiber laser device that describes an operation of the fiber laser device for cut-free transfer of the tissue of the correction area from the determined actual state into the ascertained desired state.

23. The method according to claim 22, wherein the control device is further configured for
determining visual disorder data of the human or animal eye, which describes a visual disorder,
wherein the ascertained desired state of the tissue satisfies a preset visual disorder reduction criterion, which presets that the human or animal eye with the tissue in the desired state has a visual disorder reduced compared to the determined actual state of the tissue.

24. The method according to claim 22, wherein the operation described by the control data utilizes a laser-induced change of a refractive index (LIRIC) and/or a cross-linking method.

25. The method according to claim 22, wherein the control device is configured to cause the fiber laser device to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz.

26. The method according to claim 22, wherein the control device transmits the control data to the fiber laser device of the treatment apparatus.

27. A computer program including instructions that cause a control device of a treatment apparatus, having a fiber oscillator and/or a fiber amplifier, to execute a method according to claim 22.

28. A control device for providing control data to a fiber laser device for a correction of an eye tissue, the fiber laser device including a fiber oscillator and/or a fiber amplifier, wherein the control device is configured to
determine an actual state of a tissue of a correction area of a human or animal eye,
ascertain a desired state of the tissue based on the determined actual state of the tissue, and
provide control data to the fiber laser device that describes an operation of the fiber laser device for cut-free transfer of the tissue of the correction area from the determined actual state into the ascertained desired state.

29. The control device according to claim 28, wherein the control device is configured to determine visual disorder data of the human or animal eye, which describes a visual disorder, wherein the ascertained desired state of the tissue satisfies a preset visual disorder reduction criterion, which presets that the eye with the tissue in the desired state has a visual disorder reduced compared to the determined actual state of the tissue.

30. The control device according to claim 28, wherein the operation described by the control data utilizes a laser-induced change of a refractive index (URIC) and/or a cross-linking method.

31. The control device according to claim 28, wherein the control device is configured to cause the fiber laser device to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz.

32. The control device according to claim 28, wherein the control device transmits the control data to the fiber laser device.

33. A non-transitory computer readable storage medium storing one or more programs configured to be executed by a processor, the one or more programs comprising instructions for providing control data to a fiber laser device for a correction of an eye tissue, which includes a fiber oscillator and/or a fiber amplifier, the instructions comprising
determining an actual state of a tissue of a correction area of a human or animal eye,
ascertaining a desired state of the tissue based on the determined actual state of the tissue, and
providing control data to the fiber laser device that describe an operation of the fiber laser device for cut-free transfer of the tissue of the correction area from the determined actual state into the ascertained desired state.

34. The non-transitory computer readable storage medium according to claim 33, wherein the one or more programs comprise instructions for determining visual disorder data of the human or animal eye, which describes a visual disorder, wherein the ascertained desired state of the tissue satisfies a preset visual disorder reduction criterion, which presets that the human or animal eye with the tissue in the desired state has a visual disorder reduced compared to the determined actual state of the tissue.

35. The non-transitory computer readable storage medium according to claim 33, wherein the operation described by the control data utilizes a laser-induced change of a refractive index (LIRIC) and/or a cross-linking method.

36. The non-transitory computer readable storage medium according to claim 33, wherein the one or more programs comprise instructions for causing the fiber laser device to emit laser pulses in a wavelength range between 300 nm and 1400 nm, preferably between 700 nm and 1200 nm, at a respective pulse duration between 1 fs and 1 ns, preferably between 10 fs and 10 ps, and a repetition frequency of greater than 10 kHz, preferably between 100 kHz and 100 MHz.

37. The non-transitory computer readable storage medium according to claim 33, wherein the one or more programs comprise instructions for transmitting the provided control data to the fiber laser device.
